(19) United States
(12) Patent Application Publication
(10) Pub. No.: US 2004/0050683 A1
(43) Pub. Date: Mar. 18, 2004

(54) Method for photochemical sulfochlorination of gaseous alkanes

(76) Inventor: Jean Ollivier, Arudy (FR)

Correspondence Address: SMITH, GAMBRELL & RUSSELL, LLP, 1850 M STREET, N.W., SUITE 800, WASHINGTON, DC, 20036, US

(21) Appl. No.: 10/432,714

(22) PCT Filed: Oct. 11, 2001
(86) PCT No.: PCT/FR01/03143

(30) Foreign Application Priority Data
Nov. 27, 2000 (FR) 00/15260

(51) Int. Cl. C07C 1/00

(52) U.S. Cl. 204/157.79

(57) ABSTRACT

The invention concerns a method for making an alkanesulponyl chloride by photochemical reaction of an alkane with chlorine and sulphur dioxide, which consists in using as light source an indium-doped medium-pressure mercury lamp.

The present invention relates to the field of alkanesulfonyl chlorides and has more particularly as subject matter the manufacture of these compounds by photochemical sulfochlorination of gaseous alkanes at ambient temperature.

Given the industrial usefulness of alkanesulfonyl chlorides, in particular of methanesulfonyl chloride, the manufacture of these compounds has formed the subject of several processes composed in particular of the photochemical sulfochlorination of alkanes with chlorine and sulfur dioxide. Among these known processes, a particularly outstanding process for the photochemical sulfochlorination of gaseous alkanes at ambient temperature, such as methane, is that disclosed in patents FR 2 578 841 and FR 2 595 095.

This process, which consists essentially in reacting a gaseous mixture of alkane, of sulfur dioxide and of chlorine in the presence of ultraviolet light supplied by a mercury vapor lamp, is characterized in that the mixture comprises a large excess of sulfur dioxide with respect to the alkane and in that liquid sulfur dioxide is injected into the reaction region in order to keep the temperature of the latter constant. A plant for carrying out this process is also disclosed in the abovementioned patents, the contents of which are incorporated here by reference.

In comparison with the photochemical processes of the prior art, described in the work by F. Asinger, “Paraffines, Chemistry and Technology”, Pergamon Press, 1968, p. 520 et seq., and in patent FR 2 246 520, the process of patents FR 2 578 841 and FR 2 595 095 exhibits the advantage of not requiring the introduction of any foreign product into the reaction medium and of forming the latter solely with its necessary constituents, namely the alkane, sulfur dioxide and chlorine. Moreover, this process makes it possible to obtain good conversions and satisfactory yields, both with respect to the alkane and with respect to the chlorine. In addition, as it contributes to better absorption of the photons by the chlorine and to very easy removal of the reaction heat, this process results in excellent quantum yields and prevents any overheating of the reaction medium.

The performance of this process was subsequently improved according to patent FR 2 777 565 by using, as light source, a mercury vapor lamp doped with gallium. It was shown that, with respect to a mercury vapor lamp of equal power, the use of such a light source makes it possible to obtain a markedly greater productive output of the reactor and an improvement in the yield and in the selectivity of the reaction.

It has now been found that this process can be further improved by using, as light source, a mercury vapor lamp doped with indium. This is because, with respect to a mercury vapor lamp doped with gallium, the use of a mercury vapor lamp doped with indium makes it possible, for the same power, to further improve the distribution of the light energy in the reactor and the productive output, the yield and the selectivity.

In addition to their better light output, the lamps doped with indium exhibit a much greater longevity than that of the lamps doped with gallium and are not subject, like the latter, to slow segregation of the doping agent in the lower parts of the lamp.

A subject matter of the invention is thus a process for the manufacture of alkanesulfonyl chlorides by photochemical reaction of an alkane with chlorine and sulfur dioxide, optionally in the presence of hydrogen chloride, characterized in that use is made, as light source, of a medium-pressure mercury vapor lamp doped with indium.

The process according to the invention is targeted more particularly at the sulfochlorination of methane, which is the most difficult alkane to sulfochlorinate, but it also applies to any alkane which is a gas under the temperature and pressure conditions chosen.

Depending on the starting alkane, the proportions of the reactants in the gas mixture subjected to the light radiation can vary between the following limits:

| | |
|---|---|
| Per mol of | Per mol of C₂ or |
| methane | higher alkane |

| | | | | |
|---|---|---|---|---|
| SO₂ | 1 to 12 | mol | 7 to 14 | mol |
| Cl₂ | 0.1 to 1 | mol | 0.1 to 1 | mol |
| HCl | 0.1 to 0.6 | mol | 0 | |

and are preferably chosen as follows:

| SO₂ | 5 to 7 | mol | 10 to 13 | mol |
|---|---|---|---|---|
| Cl₂ | 0.7 to 0.9 | mol | 0.7 to 0.9 | mol |
| HCl | 0.4 to 0.5 | mol | 0 | |

The reaction is preferably carried out under a pressure greater than atmospheric pressure. Generally, this pressure can range from 1 to 15 bar relative and is preferably between 8 and 12 bar relative.

The reaction temperature, generally between 10 and 90° C., depends on the working pressure chosen. It is, for example, approximately 60° C. for 10 bar absolute and approximately 80° C. for 15 bar absolute. As in the process disclosed in patents FR 2 578 841, FR 2 595 095 and FR 2 777 565, the temperature is kept constant by injection of liquid SO₂ into the reaction region.

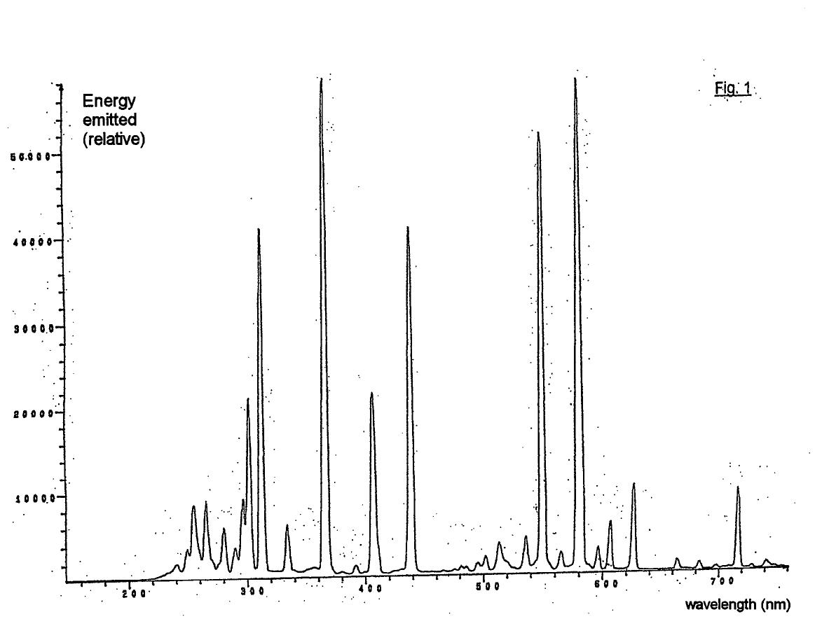

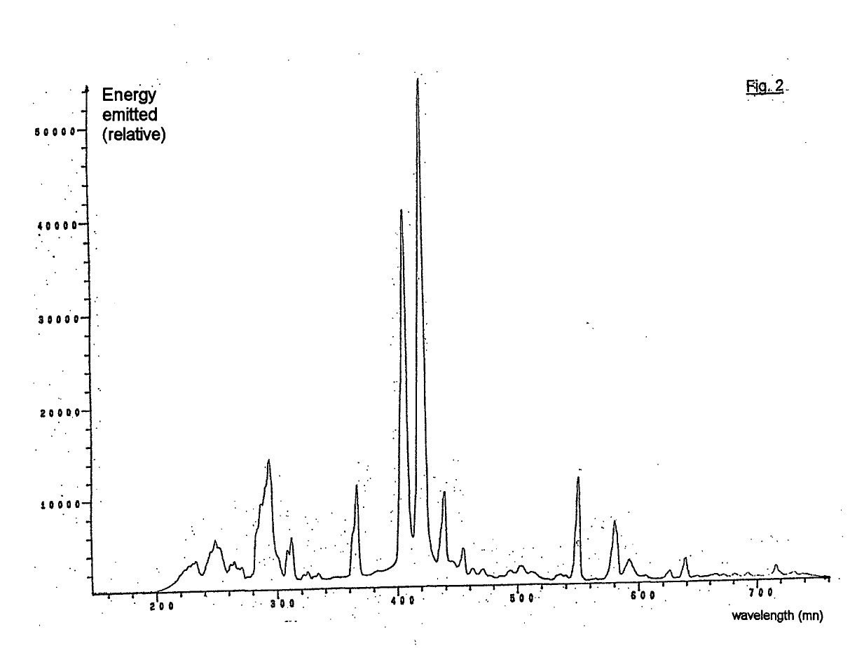

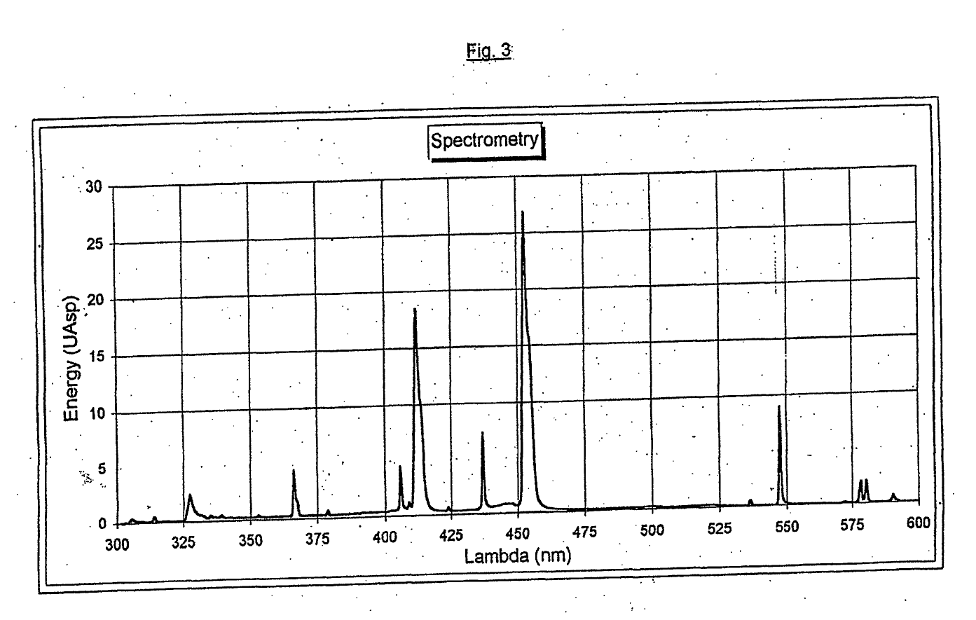

The medium-pressure mercury vapor lamps doped with indium to be used in accordance with the process according to the invention are well known and are described, for example, in the work by Mr Déribéré entitled “Lampes à Iode-Lampes à Iodures” [Iodine Lamps-Iodide Lamps], published by Dunod, 1965, p. 67, and in the work “Sources de Lumière” [Light Sources] of the Association Francaise d'Eclairage [French Lighting Association] (AFE), published by Lux, 1992, p. 134, or, finally, in “Techniques d'Utilisation des Photons” [Techniques for Using Photons] by J. C. André and A. Bernard Vannes, published by Electra/EDF, 1992, pp. 157-168. The contents of these works are incorporated here by reference. Such lamps, sold by Silitro/Scam or Heraeus, emit more than 70% of their light energy in the form of radiation with wavelengths of between 400 and 475 nm. The appended FIGS. 1, 2 and 3 respectively show the emission spectrum of a 750 watt medium-pressure mercury vapor lamp, that of a medium-pressure mercury vapor lamp of the same power doped with gallium and that of a medium-pressure mercury vapor lamp of the same power doped with indium. The light energy emitted by the medium-pressure mercury vapor lamp (FIG. 1) is distributed in the form of lines between 220 and 750 nm and that emitted by the lamp doped with gallium (FIG. 2) is between 400 and 430 nm while, for the lamp doped with indium (FIG. 3), the bulk of the energy emitted is concentrated in the region from 400 to 460 nm. In addition to a gain in working light energy efficiency (approximately 28% with respect to the gallium), the illumination of the reaction medium with a medium-pressure mercury vapor lamp doped with indium is much more homogeneous than with a conventional mercury vapor lamp. This contributes to an initiation of the reaction which is better distributed in the reaction volume and, by promoting heat transfers, makes it possible to weaken local overheatings related to the energy of the reaction; better selectivity is thus observed. With respect to the lamp doped with gallium, the productive output is improved by 23% and the selectivity with respect to the chlorine is greater than 90%.

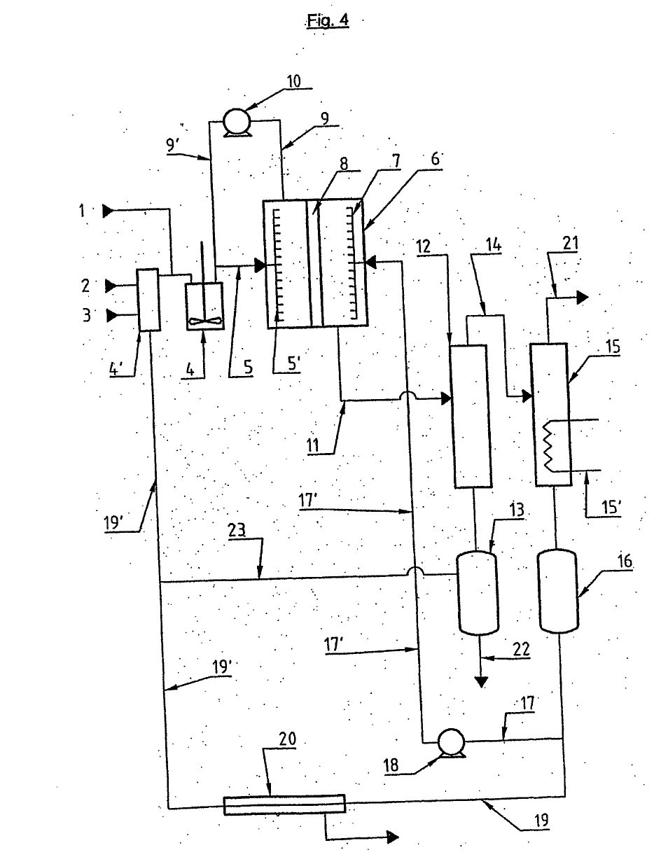

The process according to the invention can be carried out in a plant similar to that disclosed in patent FR 2 578 841. Such a plant, comprising essentially means for feeding the reactants, a photochemical reactor and means for separating the reaction products, is represented by the schematic diagram in the appended FIG. 4.

In this diagram, the inlets 1, 2 and 3 are respectively those for the alkane, sulfur dioxide and chlorine, which are introduced in the gas state into a mixer 4 equipped with a stirrer for homogenizing the gas mixture; for safety reasons, a premixer for Cl₂ and SO₂ is preferably provided at 4′. The gas mixture passes from the mixer 4 via the pipe 5 into the reactor 6, in which it is uniformly distributed by means of a perforated distribution pipe 5′. Another similar distribution pipe 7 is also positioned over the height of the reactor in order to introduce the liquid SO₂ intended for adjusting the temperature. A light source 8 passes through the reactor in a way known per se. A pipe 9 leads from the top of the reactor 6 toward a pump 10, allowing a fraction of the effluent from the reactor to be recycled toward the pipe 5 for the purpose of prediluting the reactants coming from 4. A pipe 11 conveys the liquid product, formed in the reactor 6, toward a separator 12, from where the liquid phase, that is to say the crude alkanesulfonyl chloride, descends into a holding tank 13, while the residual gases pass, via a pipe 14, into a second separator 15. This separator is optionally equipped with a cooler 15′ to bring the incoming SO₂ to the liquid state; the liquid SO₂, comprising chlorine, is recovered in a holding tank 16. An SO₂ fraction is recycled by the pipes 17 and 17′, via the pump 18 and the distribution pipe 7, to the reactor 6. Another SO₂ fraction, coming from 16, passes via the pipe 19 into the reheater 20 and from there, via 19′, toward the feed of the mixer 4.

The HCl is discharged from the top of the separator 15 via the pipe 21 toward treatment devices, which are not represented. A pipe 22 leads from the bottom of the holding tank 13 toward devices for the purification of the alkanesulfonyl chloride produced, which devices, not forming the subject matter of the invention, are not represented here.

The following examples illustrate the invention without limiting it.

### EXAMPLE 1 (COMPARATIVE)

Methanesulfonyl chloride (CH₃SO₂Cl) was prepared in the device described above using a medium-pressure mercury vapor lamp as light source. This lamp, with a power of 750 watts, was positioned axially in a reactor 6 with a capacity of 50 liters.

The gas mixture prepared in 4 comprised, per mole of methane, 6.25 mol of sulfur dioxide, 0.83 mol of chlorine and 0.417 mol of hydrogen chloride. This gas mixture was fed to the reactor at the flow rate of 5.75 Sm^{3/}hour. The pressure in the reactor being set at 9 bar above atmospheric pressure, the temperature was adjusted to 65≅2° C. by injection, by means of the distribution pipe 7, of 5.1 kg/h of liquid SO₂.

The hourly amount of crude methanesulfonyl chloride, collected after reduction in pressure in the tank 13, was 2.5 kg. At atmospheric pressure and ambient temperature, this crude product exhibited the following composition by weight:

| Constituent | Weight % |
|---|---|
| CH₃SO₂Cl | 76.5 |
| SO₂ | 18.4 |
| CH₃Cl | 0.5 |
| CH₂Cl₂ | 1.5 |
| CHCl₃ | 2.0 |
| CCl₄ | 0.1 |
| Heavy products | 1 |

The gaseous effluent arriving via 14 in the second separator exhibited the following composition by volume:

| Constituent | Volume % |
|---|---|
| SO₂ | 83.06 |
| CH₄ | 4.33 |
| HCl | 11.1 |
| Cl₂ | 1.0 |
| CH₃Cl | 0.5 |

The flow rate of this gaseous effluent was 6.57 Sm³/h and comprised the gaseous SO₂ resulting from the evaporation which served to cool the reaction. In order to collect the sulfur dioxide in the liquid state under a relative pressure of 4 bar, the temperature in the separator 15 was kept below 32° C.

The methane flow rate at the outlet 21 of the separator 15 was 0.278 Sm³/h. As the amount introduced at 1 was 0.68 Sm³/h, the conversion of the methane was therefore 59%. For the chlorine, the conversion amounted to 88%.

The results led to the following yields of and selectivity for methanesulfonyl chloride produced:

| | Yield (%) | Selectivity (%) |
|---|---|---|
| With respect to CH₄ | 55 | 93 |
| With respect to Cl₂ | 70 | 80.6 |

With regard to the power of the medium-pressure mercury vapor lamp, the methanesulfonyl chloride productive output was 2.55 kg/kW.

### EXAMPLE 2 (COMPARATIVE)

Methanesulfonyl chloride was prepared in the same equipment as for example 1, the conventional mercury vapor lamp being replaced by a lamp doped with gallium of the same electrical power (750 W).

In order to have the same degree of conversion of the chlorine as in example 1 (88%), the hourly flow rate of the feed gas mixture had to be brought to 6.86 Sm³/hour. The pressure in the reactor being set at 9 bar above atmospheric pressure, the temperature was adjusted to 65±2° C. by injection, by means of the distribution pipe 7, of 7.5 kg/h of liquid sulfur dioxide.

The hourly amount of crude methanesulfonyl chloride, collected after reduction in pressure in the tank 13, was 3.54 kg. At atmospheric pressure and at ambient temperature, this crude product exhibited the following composition by weight:

| Constituent | Weight % |
|---|---|
| CH₃SO₂Cl | 76 |
| SO₂ | 21.15 |
| CH₃Cl | 0.4 |
| CH₂Cl₂ | 0.6 |
| CHCl₃ | 0.8 |
| CCl₄ | 0.05 |
| Heavy products | 1 |

The gaseous effluent arriving via 14 in the second separator exhibited the following composition by volume:

| Constituent | Volume % |
|---|---|
| SO₂ | 84.6 |
| CH₄ | 3.17 |
| HCl | 10.81 |
| Cl₂ | 0.92 |
| CH₃Cl | 0.5 |

The flow rate of this gaseous effluent, comprising the gaseous SO₂ resulting from the evaporation which served to cool the reaction, was 8.3 Sm³/h. In order to collect the sulfur dioxide in the liquid state under a relative pressure of 4 bar, the temperature in the separator 15 was kept below 32° C.

The methane flow rate at the outlet 21 of the separator 15 was 0.26 Sm³/hour. As the amount introduced at 1 was 0.8 Sm³/h, the conversion of the methane was therefore 67%. For the chlorine, the conversion amounted to 88%.

The results led to the following yields of and selectivities for methanesulfonyl chloride produced:

| | Yield (%) | Selectivity (%) |
|---|---|---|
| With respect to CH₄ | 64.3 | 95.5 |
| With respect to Cl₂ | 76 | 86.4 |

With regard to the power of the lamp with gallium, the productive output of methanesulfonyl chloride was 3.58 kg/kW.

### EXAMPLE 3

Methanesulfonyl chloride was prepared in the same equipment as for example 1, the conventional mercury vapor lamp being replaced by a lamp doped with indium of the same electrical power (750 W).

In order to have the same degree of conversion of the chlorine as in example 1 (88%), the hourly flow rate of the feed gas mixture had to be brought to 8.82 Sm³/hour. The pressure in the reactor being set at 9 bar above atmospheric pressure, the temperature was adjusted to 65±2° C. by injection, by means of the distribution pipe 7, of 9.64 kg/h of liquid sulfur dioxide.

The hourly amount of crude methanesulfonyl chloride, collected after reduction in pressure in the tank 13, was 4.55 kg. At atmospheric pressure and at ambient temperature, this crude product exhibited the following composition by weight:

| Constituent | Weight % |
|---|---|
| CH₃SO₂Cl | 76.5 |
| SO₂ | 21.0 |
| CH₃Cl | 0.2 |
| CH₂Cl₂ | 0.4 |
| CHCl₃ | 0.4 |
| CCl₄ | 0.025 |
| Heavy products | 1 |

The gaseous effluent arriving via 14 in the second separator 15 exhibited the following composition by volume:

| Constituent | Volume % |
|---|---|
| SO₂ | 78.4 |
| CH₄ | 4.6 |
| HCl | 15.2 |
| Cl₂ | 1.3 |
| CH₃Cl | 0.5 |

The flow rate of this gaseous effluent, comprising the gaseous SO₂ resulting from the evaporation which served to cool the reaction, was 7.49 Sm³/h. In order to collect the sulfur dioxide in the liquid state under a relative pressure of 4 bar, the temperature in the separator 15 was kept below 32° C. The methane flow rate at the outlet 21 of the separator 15 was 0.326 Sm³/hour. As the amount introduced at 1 was 1.038 Sm³/h, the conversion of the methane was therefore 68.6%. For the chlorine, the conversion amounted to 88%.

The results led to the following yields of and selectivities for methanesulfonyl chloride produced:

| | Yield (%) | Selectivity (%) |
|---|---|---|
| With respect to CH₄ | 65.7 | 98.2 |
| With respect to Cl₂ | 81 | 91.6 |

With regard to the power of the lamp with indium, the productive output of methanesulfonyl chloride was 4.65 kg/kW.

The results of the preceding examples are summarized in the following table:

| | EXAMPLE 1 | EXAMPLE 2 | EX- |
|---|---|---|---|
| | (Comparative) | (Comparative) | AMPLE 3 |
| Light source | Hg lamp | Ga lamp | In lamp |
| CH₄ conversion | 59% | 67% | 68% |
| Cl₂ conversion | 88% | 88% | 88% |
| CH₃SO₂Cl yield: | | | |
| with respect to CH₄ | 55% | 64.3% | 65.7% |
| with respect to Cl₂ | 70% | 76% | 81% |
| CH₃SO₂Cl selectivity: | | | |
| with respect to CH₄ | 93% | 95.5% | 98.2% |
| with respect to Cl₂ | 80.6% | 86.4% | 91.6% |
| CH₃SO₂Cl productive | 2.55 | 3.58 | 4.65 |
| output (kg/kW) | | | |

1. A process for the manufacture of alkanesulfonyl chlorides by photochemical reaction of an alkane with chlorine and sulfur dioxide, optionally in the presence of hydrogen chloride, characterized in that use is made, as light source, of a medium-pressure mercury vapor lamp doped with indium.

2. The process as claimed in claim 1, which is carried out under a pressure ranging from 1 to 15 bar relative, preferably of between 8 and 12 bar relative.

3. The process as claimed in claim 1 or 2, wherein the reaction temperature is between 10 and 90° C. and is kept constant by injection of liquid SO₂ into the reaction region.

4. The process as claimed in one of claims 1 to 3, wherein the alkane is methane, the gas mixture fed to the reactor comprising 1 to 12 mol of sulfur dioxide, 0.1 to 1 mol of chlorine and 0.1 to 0.6 mol of hydrogen chloride per mole of methane.

5. The process as claimed in claim 4, wherein the gas mixture comprises 5 to 7 mol of sulfur dioxide, 0.7 to 0.9 mol of chlorine and 0.4 to 0.5 mol of hydrogen chloride per mole of methane.

6. The process as claimed in one of claims 1 to 3, wherein the alkane comprises at least 2 carbon atoms, the gas mixture fed to the reactor comprising 7 to 14 mol of sulfur dioxide and 0.1 to 1 mol of chlorine per mole of alkane.

7. The process as claimed in claim 6, wherein the gas mixture comprises 10 to 13 mol of sulfur dioxide and 0.7 to 0.9 mol of chlorine per mole of alkane.
